(19) United States
(12) Patent Application Publication
(10) Pub. No.: US 2025/0174384 A1
(43) Pub. Date: May 29, 2025

(54) MULTI-CHANNEL COIL CONTROL DEVICE

(71) Applicant: Metal Industries Research & Development Centre, Kaohsiung (TW)

(72) Inventors: I-Chiao Tsai, Kaohsiung City (TW); Po-Chi Hu, Kaohsiung City (TW); Wen-Hui Huang, Kaohsiung City (TW); Jia Yan Lin, Kaohsiung City (TW); Zong-Hsin Liu, Kaohsiung City (TW); Chih-Hsuan Huang, Kaohsiung City (TW); Chih-Chung Lin, Kaohsiung City (TW)

(73) Assignee: Metal Industries Research & Development Centre, Kaohsiung (TW)

(21) Appl. No.: 18/808,062

(22) Filed: Aug. 18, 2024

(30) Foreign Application Priority Data
Nov. 23, 2023 (TW) 112145344

(51) Int. Cl. H01F 7/06; H03K 17/56

(52) U.S. Cl. CPC H01F 7/064; H03K 17/56

(57) ABSTRACT

A multi-channel coil control device includes a power supply, a multi-axis coil module, and a multi-channel control circuit, and is coupled to the power supply and multi-axis coil module. The multi-channel control circuit includes an anti-reverse flow module, a charge and discharge module, a high-power switching module, and a micro control module. The anti-reverse flow module is coupled to power supply. The charge and discharge module is coupled to anti-reverse flow module. The high-power switching module is coupled to charge and discharge module. The micro control module is coupled to the power supply, anti-reverse flow module, charge and discharge module, and high-power switching module, and is used to control the anti-reverse flow module, charge and discharge module, and high-power switching module to generate pulse current signals according to power signals provided by power supply, so that the multi-channel coil module generates a magnetic stimulation signal according to pulse current signals.

### CROSS-REFERENCE TO RELATED APPLICATION

This application claims the priority benefit of Taiwan application serial no. 112145344, filed on Nov. 23, 2023. The entirety of the above-mentioned patent application is hereby incorporated by reference herein and made a part of this specification.

### BACKGROUND

### Technical Field

The disclosure relates to a control device, and in particular to a multi-channel coil control device.

### Description of Related Art

The conventional multi-channel coil driving circuit has complex circuit design and mutual interference between multiple driving components, which causes the magnetic field generated by the coil to be unstable. Also, the circuit is limited by the quantity of coils and lacks scalability.

### SUMMARY

The disclosure provides a multi-channel coil control device, which can effectively drive at least one of multiple coils to generate a magnetic field.

The multi-channel coil control device of the disclosure includes a power supply, a multi-axis coil module, and a multi-channel control circuit. The multi-channel control circuit is coupled to the power supply and the multi-axis coil module. The multi-channel control circuit includes an anti-reverse flow module, a charge and discharge module, a high-power switching module, and a micro control module. The anti-reverse flow module is coupled to the power supply. The charge and discharge module is coupled to the anti-reverse flow module. The high-power switching module is coupled to the charge and discharge module. The micro control module is coupled to the power supply, the anti-reverse flow module, the charge and discharge module, and the high-power switching module. The micro control module is used to control the anti-reverse flow module, the charge and discharge module, and the high-power switching module to generate multiple pulse current signals according to a power signal provided by the power supply, so that the multi-axis coil module generates a magnetic field according to the multiple pulse current signals.

Based on the above, the multi-channel coil control device of the disclosure can generate the multiple pulse current signals by controlling the anti-reverse flow module, the charge and discharge module, and the high-power switching module, so that the multi-axis coil module generates the magnetic field according to the multiple pulse current signals and generates a corresponding magnetic stimulation electric field on a treatment object.

In order to make the above-mentioned features and advantages of the disclosure more comprehensible, embodiments are given below and described in detail with reference to the accompanying drawings.

### BRIEF DESCRIPTION OF THE DRAWINGS

FIG. 1A to FIG. 1D are different views of a transcranial magnetic stimulation three-dimensional coil device according to an embodiment of the disclosure.

FIG. 2 shows schematic views of different transcranial magnetic stimulation electric fields according to the embodiments of FIG. 1A to FIG. 1D of the disclosure.

FIG. 3A and FIG. 3B are different views of a transcranial magnetic stimulation three-dimensional coil device according to an embodiment of the disclosure.

FIG. 4 is a schematic view of the transcranial magnetic stimulation three-dimensional coil device of FIG. 3A and FIG. 3B of the disclosure.

FIG. 5 is a schematic circuit view of a multi-channel coil control device according to an

embodiment of the disclosure.

FIG. 6 is a schematic circuit view of a multi-channel coil control device according to an embodiment of the disclosure.

FIG. 7 is a schematic circuit view of a multi-channel coil control device according to an embodiment of the disclosure.

### DESCRIPTION OF THE EMBODIMENTS

In order to make the content of the disclosure more comprehensible, the following embodiments are provided as examples of implementing the disclosure accordingly. In addition, wherever possible, elements/components/steps with the same reference numerals in the drawings and embodiments may represent the same or similar parts.

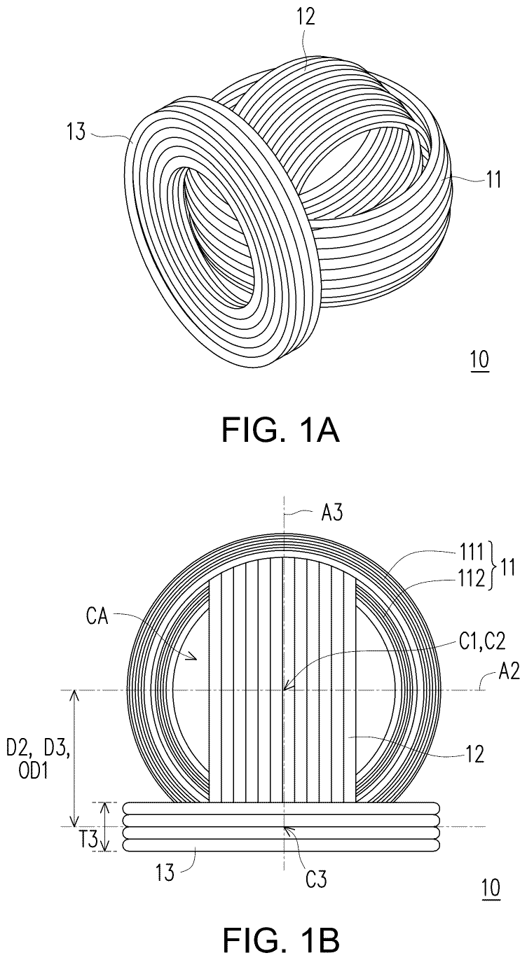

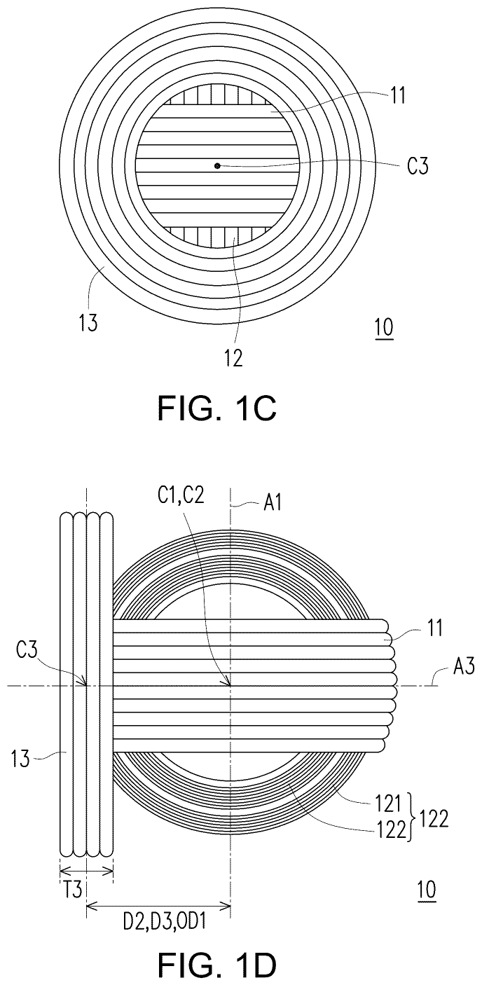

FIG. 1A to FIG. 1D are different views of a transcranial magnetic stimulation three-dimensional coil device according to an embodiment of the disclosure. Referring to FIG. 1A to FIG. 1D, a transcranial magnetic stimulation three-dimensional coil device 10 includes a first coil 11, a second coil 12, and a third coil 13. The first coil 11 has a first center axis A1 and a center area CA. The second coil 12 has a second center axis A2, and the second coil 12 is located in the center area CA. The third coil 13 has a third center axis A3 and is located on a side of the first coil 11, in which the first center axis A1, the second center axis A2, and the third center axis A3 are perpendicular to each other.

In FIG. 1B, the first coil 11 has an outer layer sub-coil 111 and an inner layer sub-coil 112, and the second coil 12 is between the outer layer sub-coil 111 and the inner layer sub-coil 112. In addition, in FIG. 1D, the second coil 12 has an outer layer sub-coil 121 and an inner layer sub-coil 122, and the outer layer sub-coil 121 of the second coil 12 is between the outer layer sub-coil 111 and the inner layer sub-coil 112, and the inner layer sub-coil 112 of the first coil 11 is between the outer layer sub-coil 121 and the inner layer sub-coil 122 of the second coil 12, but the disclosure is not limited thereto.

In an embodiment, the first coil 11 has a first center point C1, the second coil 12 has a second center point C2, and a first distance between the first center point C1 and the second center point C2 is not greater than a first threshold value. In FIG. 1B and FIG. 1D, the first center point C1 may coincide with/be the same as the second center point C2, for example. In different embodiments, the first threshold value may be set to any value that may be considered to make the first center point C1 sufficiently close to the second center point C2 according to the needs of the designer, and the disclosure is not limited thereto.

In an embodiment, the third coil 13 has a third center point C3, and a second distance D2 between the third center point C3 and the first center point C1 or a third distance D3 between the third center point C3 and the second center point C2 is within a preset range, in which the preset range may be based on an outer diameter OD1 of the first coil 11 and a thickness T3 of the third coil 13. In an embodiment, the upper limit of the preset range is, for example, the sum of the outer diameter OD1 and half of the thickness T3, and the lower limit of the preset range is, for example, the outer diameter OD1 minus half of the thickness T3. However, the disclosure is not limited thereto.

In FIG. 1A to FIG. 1D, the first center point C1 may coincide with the second center point C2, and the second distance D2 between the third center point C3 and the first center point C1 may be equal to the third distance D3 between the third center point C3 and the second center point C2, but the disclosure is not limited thereto. In addition, the outer diameter OD1 may be equal to the second distance D2 (and the third distance D3), but the disclosure is not limited thereto.

In an embodiment, the quantity of layers of the first coil 11 is 2, and the quantity of turns of each layer of the first coil 11 is 10 turns or 8 turns. In an embodiment, the quantity of layers of the second coil 12 is 2, and the quantity of turns of each layer of the second coil 12 is 12 turns or 10 turns. In an embodiment, the quantity of layers of the third coil 13 is 4, and the quantity of turns of each layer of the third coil 13 is 6 turns, 5 turns, 4 turns, or 3 turns.

In an embodiment, the coil diameter of at least one of the first coil 11, the second coil 12, and the third coil 13 is less than 3.5 mm (millimeter). In an embodiment, the coil resistance of at least one of the first coil 11, the second coil 12, and the third coil 13 is less than 45 ohm. In an embodiment, the coil inductance of at least one of the first coil 11, the second coil 12 and the third coil 13 is less than μH (microhenry).

In an embodiment, at least one of the first coil 11, the second coil 12, and the third coil 13 may be made of Ritz wire. In an embodiment, the wire diameter of at least one of the first coil 11, the second coil 12, and the third coil 13 is 30 American wire gauge.

In this embodiment, the first coil 11 may be driven to generate a first magnetic field, the second coil 12 may be driven to generate a second magnetic field, and the third coil 13 may be driven to generate a third magnetic field. In an embodiment, at least one of the first coil 11, the second coil 12, and the third coil 13 may be driven to generate a magnetic field and may generate a corresponding magnetic stimulation electric field on a treatment object. The treatment object may be, for example, a human head, and the magnetic stimulation electric field may be a transcranial magnetic stimulation (TMS) electric field.

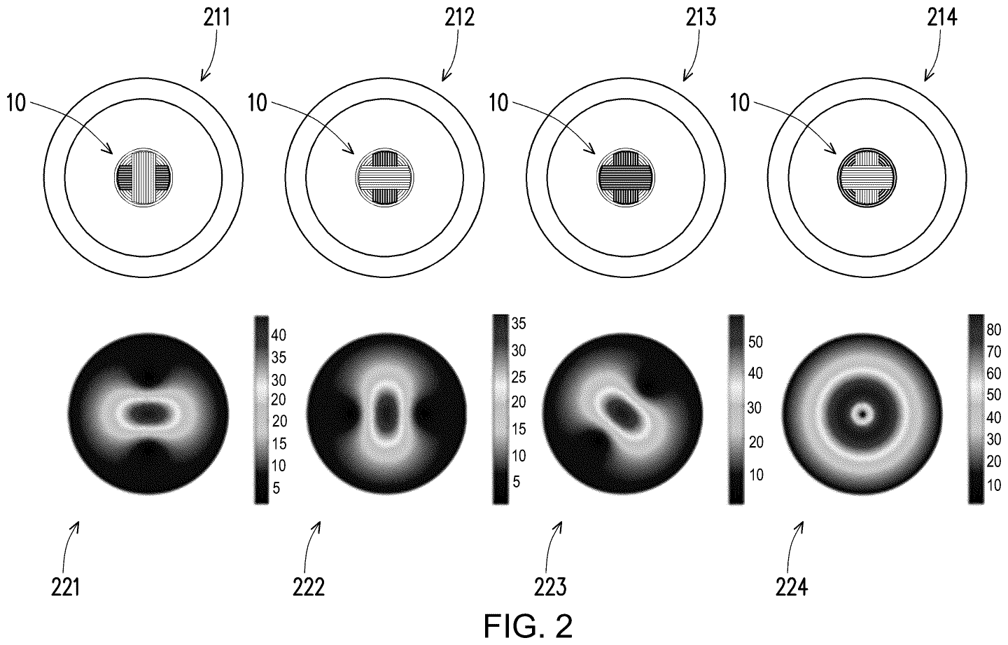

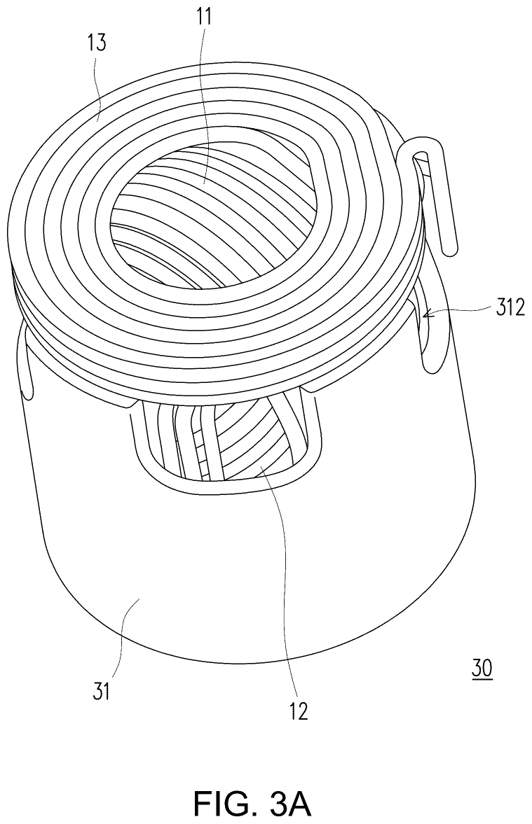

FIG. 2 shows schematic views of different transcranial magnetic stimulation electric fields according to the embodiments of FIG. 1A to FIG. 1D of the disclosure. Referring to FIG. 1A to FIG. 2, a treatment scenario 211 is, for example, a scenario in which only the first coil 11 of the transcranial magnetic stimulation three-dimensional coil device 10 is excited to provide the first magnetic field. Since the second coil 12 and the third coil 13 are not excited, a transcranial magnetic stimulation electric field 221 provided by the transcranial magnetic stimulation three-dimensional coil device 10 is only formed by the first magnetic field of the first coil 11. On the other hand, a treatment scenario 212 is, for example, a scenario in which only the second coil 12 of the transcranial magnetic stimulation stereoscopic coil device 10 is excited to provide the second magnetic field. Since the first coil 11 and the third coil 13 are not excited, a transcranial magnetic stimulation electric field 222 provided by the transcranial magnetic stimulation three-dimensional coil device 10 is only formed by the second magnetic field of the second coil 12.

A treatment scenario 213 is, for example, a scenario in which the first coil 11 and the

second coil 12 of the transcranial magnetic stimulation three-dimensional coil device 10 are simultaneously excited to respectively provide the first magnetic field and the second magnetic field. Since the third coil 13 is not excited, a transcranial magnetic stimulation electric field 223 provided by the transcranial magnetic stimulation three-dimensional coil device 10 may be generated by cooperated operation of the first magnetic field of the first coil 11 and the second magnetic field of the second coil 12.

It may be seen from FIG. 2 that by simultaneously exciting the first coil 11 and the second coil 12, the transcranial magnetic stimulation electric field 223 may be rotated accordingly. In this case, the transcranial magnetic stimulation electric field 223 may be rotated accordingly according to the needs of the designer by simply adjusting the current intensity used to excite the first coil 11 and/or the second coil 12.

A treatment scenario 214 is, for example, a scenario in which only the third coil 13 of the transcranial magnetic stimulation three-dimensional coil device 10 is excited to provide the third magnetic field. Since the first coil 11 and the second coil 12 are not excited, a transcranial magnetic stimulation electric field 224 provided by the transcranial magnetic stimulation three-dimensional coil device 10 is only formed by the third magnetic field of the third coil 13.

It may be seen from the transcranial magnetic stimulation electric fields 223, 224 that if the third coil 13 is excited while the first coil 11 and the second coil 12 are excited, the transcranial magnetic stimulation electric field 223 may be shifted left/right accordingly.

It may be seen that the transcranial magnetic stimulation electric field 223 may be rotated

and displaced according to the needs of the designer by simply adjusting the current intensity used to excite the first coil 11, the second coil 12 and/or the third coil 13, then the formed transcranial magnetic stimulation electric field presents configurations/manners required by the designer. In this way, the transcranial magnetic stimulation three-dimensional coil device 10 of the disclosure may treat different brain areas of a patient through the transcranial magnetic stimulation electric field having different configurations/manners without changing the coil.

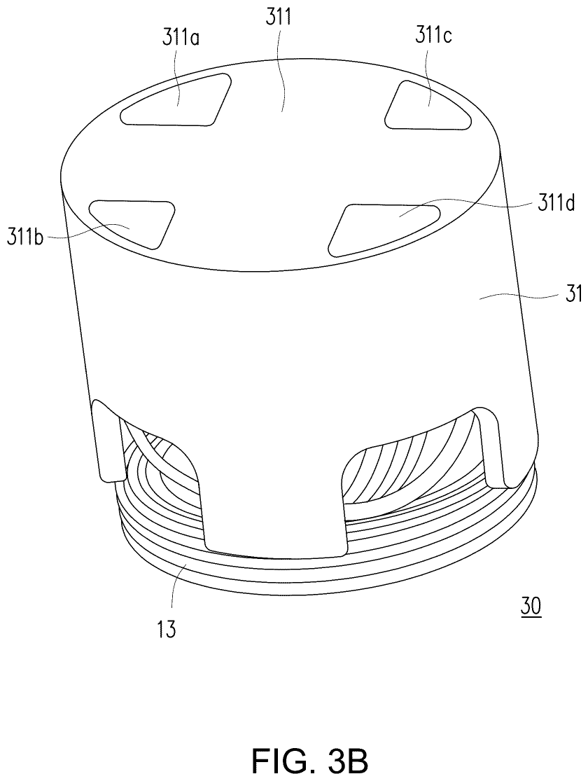

FIG. 3A and FIG. 3B are different views of a transcranial magnetic stimulation three-dimensional coil device according to an embodiment of the disclosure. Referring to FIG. 3A and FIG. 3B, in this embodiment, a transcranial magnetic stimulation three-dimensional coil device 30 includes respective components of the transcranial magnetic stimulation three-dimensional coil device 10 of FIG. 1A to FIG. 1D (for example, the first coil 11, the second coil 12, and the third coil 13), and further includes a cylindrical housing 31, which is used to accommodate the first coil 11 and the second coil 12. In FIG. 3A and FIG. 3B, the cylindrical housing 31 has a bottom surface 311 and an opening 312 opposite to the bottom surface, in which the bottom surface 311 is disposed with ventilation holes 311a to 311d, and the opening 312 is used to be sleeved on the first coil 11 and the second coil 12.

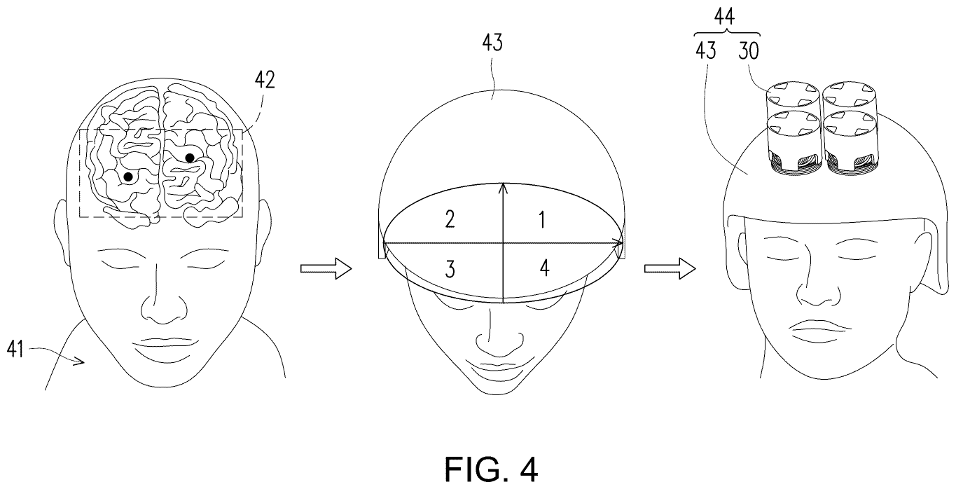

FIG. 4 is a schematic view of the transcranial magnetic stimulation three-dimensional coil device of FIG. 3A and FIG. 3B of the disclosure. In FIG. 4, assuming that a brain area 42 of a patient 41 is determined/diagnosed to require treatment via TMS technology, then relevant personnel (such as doctors) may plan designated positions used to dispose one or more transcranial magnetic stimulation three-dimensional coils 30 on a wearable device 43 (such as a helmet to be worn by the patient 41), for example, positions marked with given numbers 1 to 4 in FIG. 4, but the disclosure is not limited thereto.

Based on above, several transcranial magnetic stimulation three-dimensional coils 30 may be disposed at corresponding designated positions. Afterward, each transcranial magnetic stimulation three-dimensional coil device 30 is excited to provide a transcranial magnetic stimulation electric field, and the respective transcranial magnetic stimulation electric fields corresponding to the transcranial magnetic stimulation three-dimensional coil devices 30 cooperate to form an integrated transcranial magnetic stimulation electric field corresponding to a transcranial magnetic stimulation three-dimensional coil device 44 (which includes the wearable device 43 and the one or more transcranial magnetic stimulation three-dimensional coil devices 30). In this way, the transcranial magnetic stimulation three-dimensional coil device 44 of the disclosure may treat different brain areas of the patient through the integrated transcranial magnetic stimulation electric field having different configurations/manners without changing the coil.

In this embodiment, the transcranial magnetic stimulation three-dimensional coil device 30 includes three coils having center axes perpendicular to each other. The transcranial magnetic stimulation three-dimensional coil device 30 can generate a corresponding magnetic field by simply adjusting the current intensity used to excite the coil, and the magnetic field can generate a corresponding transcranial magnetic stimulation electric field in the human head, in which the transcranial magnetic stimulation electric field may present corresponding rotation and displacement according to the needs of the designer, thereby the transcranial magnetic stimulation electric field provided by the transcranial magnetic stimulation three-dimensional coil device 30 may present the configurations/manners required by the designer. In other words, the transcranial magnetic stimulation three-dimensional coil device 30 may treat different brain areas of the patient through the transcranial magnetic stimulation electric field having different configurations/manners without changing the coil.

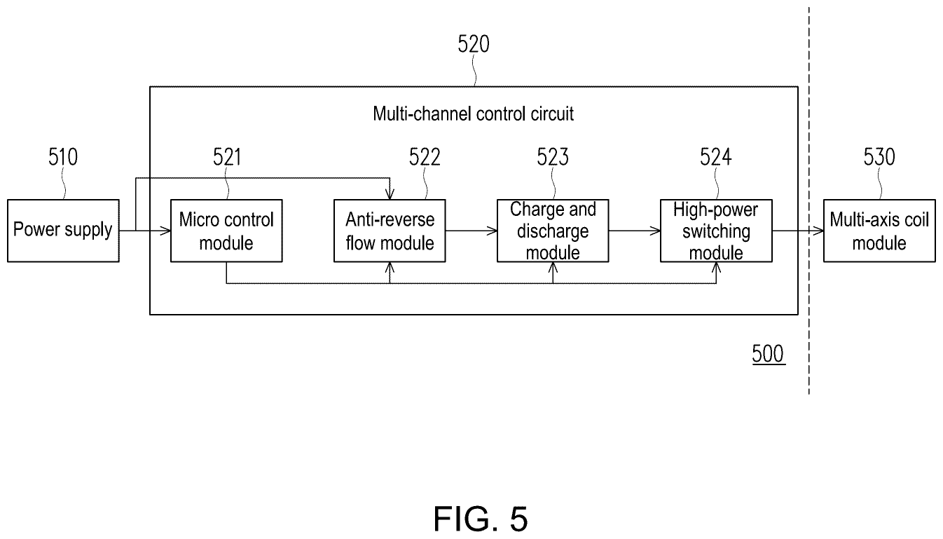

FIG. 5 is a schematic circuit view of a multi-channel coil control device according to an embodiment of the disclosure. Referring to FIG. 5, a multi-channel coil control device 500 includes a power supply 510 and a multi-channel control circuit 520, in which the multi-channel control circuit 520 may drive a multi-axis coil module 530. It should be noted that the multi-axis coil module 530 of this embodiment may be the transcranial magnetic stimulation three-dimensional coil devices 10, 30 described in the embodiments of FIG. 1A to FIG. 4, but the disclosure is not limited thereto. In this embodiment, the multi-channel control circuit 520 includes a micro control module 521, an anti-reverse flow module 522, a charge and discharge module 523, and a high-power switching module 524. The power supply 510 is coupled to the micro control module 521 and the anti-reverse flow module 522. The micro control module 521 is further coupled to the anti-reverse flow module 522, the charge and discharge module 523, and the high-power switching module 524. In this embodiment, the micro control module 521 is used to control the anti-reverse flow module 522, the charge and discharge module 523, and the high-power switching module 524 to generate multiple pulse current signals according to the power signal provided by the power supply 510, so that the multi-axis coil module 530 generates the corresponding magnetic field according to the pulse current signals.

In this embodiment, the multi-axis coil module 530 may include multiple coils. The multi-channel control circuit 520 may output the multiple pulse current signals to the coils to excite at least one of the coils to generate the magnetic field. In this regard, the power supply 510 may, for example, provide a direct current to the micro control module 521 and the anti-reverse flow module 522. The micro control module 521 may control multiple control switches and switching switches disposed among the anti-reverse flow module 522, the charge and discharge module 523, and the high-power switching module 524 to generate the multiple pulse current signals according to the direct current provided by the power supply 510 to the coils.

In this embodiment, the micro control module 521 may include multiple micro control units, and the micro control units are respectively coupled to the power supply 510, the anti-reverse flow module 522, the charge and discharge module 523, and the high-power switching module 524. The anti-reverse flow module 522 may include multiple anti-reverse flow units, and the anti-reverse flow units are respectively coupled between the charge and discharge module 523 and the multiple micro control units. The multiple micro control units respectively control the anti-reverse flow units. The charge and discharge module may include multiple charge and discharge units, the charge and discharge units are respectively coupled between the multiple anti-reverse flow units and the high-power switching module 524, and the multiple micro control units respectively control the charge and discharge units. The high-power switching module 524 includes multiple high-power switching units, the high-power switching units are respectively coupled between the multiple charge and discharge units and the multiple coils, and the multiple micro control units respectively control the high-power switching units. It should be noted that quantities of the micro control units, the anti-reverse flow units, the charge and discharge units, and the high-power switching units are determined according to a quantity of channels, and the quantity of channels is the quantity of coils in the multi-axis coil module 530.

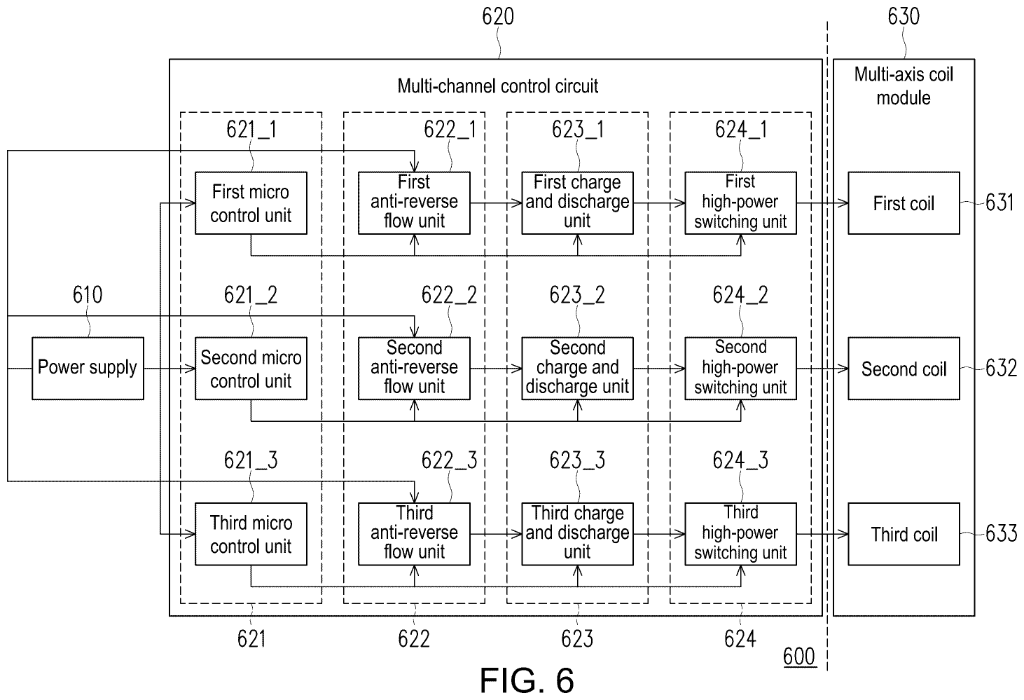

FIG. 6 is a schematic circuit view of a multi-channel coil control device according to an embodiment of the disclosure. Referring to FIG. 6, in this embodiment, a multi-axis coil module 630 has three sets of coils (that is, the quantity of channels is three), in which the multi-axis coil module 630 may include a first coil 631, a second coil 632, and a third coil 633. In this embodiment, a multi-channel coil control device 600 includes a power supply 610 and a multi-channel control circuit 620. The multi-channel control circuit 620 includes a micro control module 621, an anti-reverse flow module 622, a charge and discharge module 623, and a high-power switching module 624. The power supply 610 is coupled to the micro control module 621 and the anti-reverse flow module 622. The micro control module 621 is further coupled to the anti-reverse flow module 622, the charge and discharge module 623, and the high-power switching module 624. In this embodiment, the micro control module 621 is used to control the anti-reverse flow module 622, the charge and discharge module 623, and the high-power switching module 624 to generate multiple pulse current signals according to the power signal provided by the power supply 610, so that the multi-axis coil module 630 generates the corresponding magnetic field according to the pulse current signals.

In this embodiment, the micro control module 621 may include a first micro control unit 621_1, a second micro control unit 621_2, and a third micro control unit 621_3. The first micro control unit 621_1, the second micro control unit 621_2, and the third micro control unit 621_3 may respectively be micro control units (MCU). The anti-reverse flow module 622 may include a first anti-reverse flow unit 622_1, a second anti-reverse flow unit 622_2, and a third anti-reverse flow unit 622_3. The charge and discharge module 623 may include a first charge and discharge unit 623_1, a second charge and discharge unit 623_2, and a third charge and discharge unit 623_3. The high-power switching module 624 includes a first high-power switching unit 624_1, a second high-power switching unit 624_2, and a third high-power switching unit 624_3.

In this embodiment, the power supply 610 is coupled to the first micro control unit 621_1 and the first anti-reverse flow unit 622_1. The first micro control unit 621_1 is further coupled to the first anti-reverse flow unit 622_1, the first charge and discharge unit 623_1, and the first high-power switching unit 624_1. The first micro control unit 621_1 may control multiple control switches and switching switches among the first anti-reverse flow unit 622_1, the first charge and discharge unit 623_1, and the first high-power switching unit 624_1, so that the current signal provided by the power supply 610 may be further transmitted to the first charge and discharge unit 623_1 and the first high-power switching unit 624_1 via the first anti-reverse flow unit 622_1, and through switching operations of the multiple switching switches in the first high-power switching unit 624_1, a first pulse current signal is generated to the first coil 631. In this way, the first coil 631 can generate the corresponding first magnetic field through the first pulse current signal.

In this embodiment, the power supply 610 is coupled to the second micro control unit 621_2 and the second anti-reverse flow unit 622_2. The second micro control unit 621_2 is further coupled to the second anti-reverse flow unit 622_2, the second charge and discharge unit 623_2, and the second high-power switching unit 624_2. The second micro control unit 621_2 may control multiple control switches and switching switches among the second anti-reverse flow unit 622_2, the second charge and discharge unit 623_2, and the second high-power switching unit 624_2, so that the current signal provided by the power supply 610 may be further transmitted to the second charge and discharge unit 623_2 and the second high-power switching unit 624_2 via the second anti-reverse flow unit 622_2, and through switching operations of the multiple switching switches in the second high-power switching unit 624_2, a second pulse current signal is generated to the second coil 632. In this way, the second coil 632 can generate the corresponding second magnetic field through the second pulse current signal.

In this embodiment, the power supply 610 is coupled to the third micro control unit 621_3 and the third anti-reverse flow unit 622_3. The third micro control unit 621_3 is further coupled to the third anti-reverse flow unit 622_3, the third charge and discharge unit 623_3, and the third high-power switching unit 624_3. The third micro control unit 621_3 may control multiple control switches and switching switches among the third anti-reverse flow unit 622_3, the third charge and discharge unit 623_3, and the third high-power switching unit 624_3, so that the current signal provided by the power supply 610 may be further transmitted to the third charge and discharge unit 623_3 and the third high-power switching unit 624_3 via the third anti-reverse flow unit 622_3, and through switching operations of the multiple switching switches in the third high-power switching unit 624_3, a third pulse current signal is generated to the third coil 633. In this way, the third coil 633 can generate the corresponding third magnetic field through the third pulse current signal.

In an embodiment, the first micro control unit 621_1 may be configured as a master controller, and the second micro control unit 621_2 and the third micro control unit 621_3 may be configured as slave controllers. The multi-channel coil control device 600 may further include an input interface (such as a computer mouse, a keyboard, and/or a touch screen) and a display interface (such as a monitor). The first micro control unit 621_1 may display a user interface through the display interface, and a user may input control parameters to the first micro control unit 621_1 through the input interface, so as to set related magnetic stimulation pulse parameters, and to select at least one of the multiple channels to drive at least one of the first coil 631, the second coil 632, and the third coil 633. The first micro control unit 621_1 may further operate the second micro control unit 621_2 and the third micro control unit 621_3 according to the related magnetic stimulation pulse parameters set. The first micro control unit 621_1, the second micro control unit 621_2, and the third micro control unit 621_3 may operate independently to provide different driving modes respectively.

In this embodiment, the first high-power switching unit 624_1, the second high-power switching unit 624_2, and the third high-power switching unit 624_3 may be controlled to output at least one of the first pulse current signal, the second pulse current signal, and the third pulse current signal to the first coil 631, the second coil 632, and third coil 633, so as to excite at least one of the coils to generate the magnetic field.

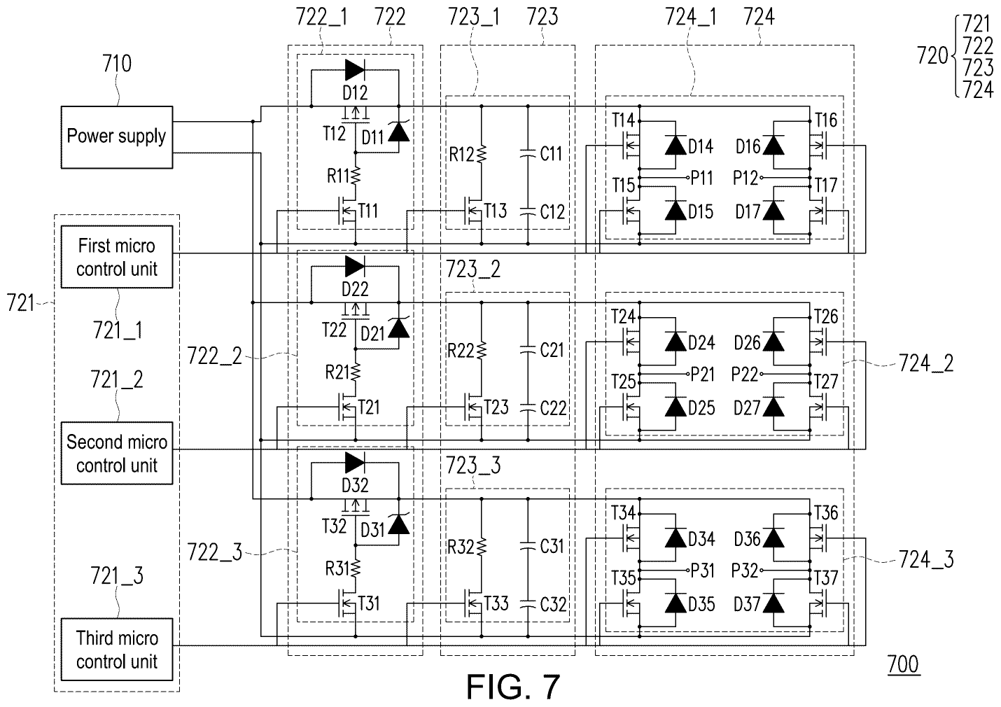

FIG. 7 is a schematic circuit view of a multi-channel coil control device according to an embodiment of the disclosure. Referring to FIG. 7, this embodiment further illustrates a circuit implementation architecture related to the above-mentioned related modules, but the disclosure is not limited thereto. In this embodiment, a multi-channel coil control device 700 includes a power supply 710 and a multi-channel control circuit 720, and the multi-channel control circuit 720 may be used to drive the first coil 631, the second coil 632, and the third coil 633 in the multi-axis coil module 630 of FIG. 6 mentioned above. In other words, the multi-channel control circuit 720 of this embodiment may have three channels.

In this embodiment, the multi-channel control circuit 720 includes a micro control module 721, an anti-reverse flow module 722, a charge and discharge module 723, and a high-power switching module 724. The power supply 710 is coupled to the micro control module 721 and the anti-reverse flow module 722. The micro control module 721 is further coupled to the anti-reverse flow module 722, the charge and discharge module 723, and the high-power switching module 724. In this embodiment, the micro control module 721 may include a first micro control unit 721_1, a second micro control unit 721_2, and a third micro control unit 721_3. The anti-reverse flow module 722 may include a first anti-reverse flow unit 722_1, a second anti-reverse flow unit 722_2, and a third anti-reverse flow unit 722_3. The charge and discharge module 723 may include a first charge and discharge unit 723_1, a second charge and discharge unit 723_2, and a third charge and discharge unit 723_3. The high-power switching module 724 includes a first high-power switching unit 724_1, a second high-power switching unit 724_2, and a third high-power switching unit 724_3.

In this embodiment, the first anti-reverse flow unit 722_1 includes control switches T11, T12, a resistor R11, and a Zener diode D11, in which there may be a parasitic diode D12 between the first terminal and the second terminal of the control switch T12. In this embodiment, the first terminal of the control switch T11 is coupled to a terminal of the resistor R11, the second terminal of the control switch T11 is coupled to the power supply 710 (for example, the negative terminal), and the control terminal of the control switch T11 is coupled to the first micro control unit 721_1. Another terminal of the resistor R11 is coupled to the control terminal of the control switch T12 and the cathode (the positive electrode) of the Zener diode D11. The first terminal of the control switch T12 is coupled to the power supply 710 (for example, the positive terminal), and the second terminal of the control switch T12 is coupled to the anode (the negative electrode) of the Zener diode D11.

In this embodiment, the first charge and discharge unit 723_1 includes a control switch T13, a resistor R12, and capacitors C11, C12. In this embodiment, the first terminal of the control switch T13 is coupled to a terminal of the resistor R12, the second terminal of the control switch T13 is coupled to the power supply 710 (for example, the negative terminal), and the control terminal of the control switch T13 is coupled to the first micro control unit 721_1. Another terminal of the resistor R12 is coupled to the first anti-reverse flow unit 722_1 and a terminal of the capacitor C11. A terminal of the capacitor C11 is coupled to the first anti-reverse flow unit 722_1, and another terminal of the capacitor C11 is coupled to a terminal of the capacitor C12. Another terminal of the capacitor C12 is coupled to the power supply 710 (for example, the negative terminal).

In this embodiment, the first high-power switching unit 724_1 includes switching switches T14 to T17, and there are parasitic diodes D14 to D17 between respective first terminals and second terminals of the switching switches T14 to T17. In this embodiment, the first terminal of the switching switch T14 is coupled to the first charge and discharge unit 723_1, the second terminal of the switching switch T14 is coupled to a first output terminal P11, and the control terminal of the switching switch T14 is coupled to the first micro control unit 721_1. The first terminal of the switching switch T15 is coupled to the first output terminal P11, the second terminal of the switching switch T15 is coupled to the power supply 710 (for example, the negative terminal), and the control terminal of the switching switch T15 is coupled to the first micro control unit 721_1. The first terminal of the switching switch T16 is coupled to the first charge and discharge unit 723_1, the second terminal of the switching switch T16 is coupled to a second output terminal P12, and the control terminal of the switching switch T16 is coupled to the first micro control unit 721_1. The first terminal of the switching switch T17 is coupled to the second output terminal P12, the second terminal of the switching switch T17 is coupled to the power supply 710 (for example, the negative terminal), and the control terminal of the switching switch T17 is coupled to the first micro control unit 721_1. The first output terminal P11 and the second output P12 may be coupled to the first coil.

In this embodiment, the control switches T11, T13 and the switching switches T14 to T17 may be high-power components respectively. The control switches T11, T13 and the switching switches T14 to T17 may be N-type transistors (such as Metal-Oxide-Semiconductor, MOS), and the control switch T12 may be a P-type transistor. The above-mentioned first terminal and the second terminal may be the source terminal and the drain terminal respectively, and the above-mentioned control terminal may be the gate terminal. In this embodiment, the first micro control unit 721_1 may output multiple control signals to the control terminals of the control switches T11 to T13 and the switching switches T14 to T17. Specifically, when the power supply 710 supplies power to charge the capacitors C11, C12 in the first charge and discharge unit 723_1, since the parasitic diode D12 causes the voltage (Vgs) between the gate terminal and the source terminal of the control switch T12 to form a negative voltage, the control switch T12 may be turned on. Also, when the first charge and discharge unit 723_1 is to discharge, the first micro control unit 721_1 may control the control switch T11 to turn off, so that the gate terminal of the control switch T12 forms an open or non-connected state, so that the control switch T12 may be in a non-conducting state. Therefore, the current released by the first charge and discharge unit 723_1 can be effectively prevented from flowing back to the power supply 710 or other circuit units. In this embodiment, the capacitors C11, C12 in the first charge and discharge unit 723_1 may provide the current to the switching switches T14 to T17. In this regard, the first micro control unit 721_1 may switch the switching switches T14 to T17 at high speeds to perform turning on and off, so that the first pulse current signal is output from the first output terminal P11 and the second output P12 to the first coil, so that the first coil can generate the corresponding electric field according to the first pulse current signal.

In this embodiment, the second anti-reverse flow unit 722_2 includes control switches T21, T22, a resistor R21, and a Zener diode D21, in which there may be a parasitic diode D22 between the first terminal and the second terminal of the control switch T22. In this embodiment, the first terminal of the control switch T21 is coupled to a terminal of the resistor R21, the second terminal of the control switch T21 is coupled to the power supply 710 (for example, the negative terminal), and the control terminal of the control switch T21 is coupled to the second micro control unit 721_2. Another terminal of the resistor R21 is coupled to the control terminal of the control switch T22 and the cathode (the positive electrode) of the Zener diode D21. The first terminal of the control switch T22 is coupled to the power supply 710 (for example, the positive terminal), and the second terminal of the control switch T22 is coupled to the anode (the negative electrode) of the Zener diode D21.

In this embodiment, the second charge and discharge unit 723_2 includes a control switch T23, a resistor R22, and capacitors C21, C22. In this embodiment, the first terminal of the control switch T23 is coupled to a terminal of the resistor R22, the second terminal of the control switch T23 is coupled to the power supply 710 (for example, the negative terminal), and the control terminal of the control switch T23 is coupled to the second micro control unit 721_2. Another terminal of the resistor R22 is coupled to the second anti-reverse flow unit 722_2 and a terminal of the capacitor C21. A terminal of the capacitor C21 is coupled to the second anti-reverse flow unit 722_2, and another terminal of the capacitor C21 is coupled to a terminal of the capacitor C22. Another terminal of the capacitor C22 is coupled to the power supply 710 (for example, the negative terminal).

In this embodiment, the second high-power switching unit 724_2 includes switching switches T24 to T27, and there are parasitic diodes D24 to D27 between respective first terminals and second terminals of the switching switches T24 to T27. In this embodiment, the first terminal of the switching switch T24 is coupled to the second charge and discharge unit 723_2, the second terminal of the switching switch T24 is coupled to the first output terminal P21, and the control terminal of the switching switch T24 is coupled to the second micro control unit 721_2. The first terminal of the switching switch T25 is coupled to the first output terminal P21, the second terminal of the switching switch T25 is coupled to the power supply 710 (for example, the negative terminal), and the control terminal of the switching switch T25 is coupled to the second micro control unit 721_2. The first terminal of the switching switch T26 is coupled to the second charge and discharge unit 723_2, the second terminal of the switching switch T26 is coupled to the second output terminal P22, and the control terminal of the switching switch T26 is coupled to the second micro control unit 721_2. The first terminal of the switching switch T27 is coupled to the second output terminal P22, the second terminal of the switching switch T27 is coupled to the power supply 710 (for example, the negative terminal), and the control terminal of the switching switch T27 is coupled to the second micro control unit 721_2. The first output terminal P21 and the second output P22 may be coupled to the second coil.

In this embodiment, the control switches T21, T23 and the switching switches T24 to T27 may be high-power components respectively. The control switches T21, T23 and the switching switches T24 to T27 may be N-type transistors, and the control switch T22 may be a P-type transistor. The above-mentioned first terminal and the second terminal may be the source terminal and the drain terminal respectively, and the above-mentioned control terminal may be the gate terminal. In this embodiment, the second micro control unit 721_2 may output multiple control signals to the control terminals of the control switches T21 to T23 and the switching switches T24 to T27. Specifically, when the power supply 710 supplies power to charge the capacitors C21, C22 in the second charge and discharge unit 723_2, since the parasitic diode D22 causes the voltage (Vgs) between the gate terminal and the source terminal of the control switch T22 to form a negative voltage, the control switch T22 may be turned on. Also, when the second charge and discharge unit 723_2 is to discharge, the second micro control unit 721_2 may control the control switch T21 to turn off, so that the gate terminal of the control switch T22 forms an open or non-connected state, so that the control switch T22 may be made in a non-conducting state. Therefore, the current released by the second charge and discharge unit 723_2 can be effectively prevented from flowing back to the power supply 710 or other circuit units. In this embodiment, the capacitors C21, C22 in the second charge and discharge unit 723_2 may provide the current to the switching switches T24 to T27. In this regard, the second micro control unit 721_2 may switch the switching switches T24 to T27 at high speeds to perform turning on and off, so that the second pulse current signal is output from the first output terminal P21 and the second output P22 to the second coil, so that the second coil can generate the corresponding electric field according to the second pulse current signal.

In this embodiment, the third anti-reverse flow unit 722_3 includes control switches T31, T32, a resistor R31, and a Zener diode D31, in which there may be a parasitic diode D32 between the first terminal and the second terminal of the control switch T32. In this embodiment, the first terminal of the control switch T31 is coupled to a terminal of the resistor R31, the second terminal of the control switch T31 is coupled to the power supply 710 (for example, the negative terminal), and the control terminal of the control switch T31 is coupled to the third micro control unit 721_3. Another terminal of the resistor R31 is coupled to the control terminal of the control switch T32 and the cathode (the positive electrode) of the Zener diode D31. The first terminal of the control switch T32 is coupled to the power supply 710 (for example, the positive terminal), and the second terminal of the control switch T32 is coupled to the anode (the negative electrode) of the Zener diode D31.

In this embodiment, the third charge and discharge unit 723_3 includes a control switch T33, a resistor R32, and capacitors C31, C32. In this embodiment, the first terminal of the control switch T33 is coupled to a terminal of the resistor R32, the second terminal of the control switch T33 is coupled to the power supply 710 (for example, the negative terminal), and the control terminal of the control switch T33 is coupled to the third micro control unit 721_3. Another terminal of the resistor R32 is coupled to the third anti-reverse flow unit 722_3 and a terminal of the capacitor C31. A terminal of the capacitor C31 is coupled to the third anti-reverse flow unit 722_3, and another terminal of the capacitor C31 is coupled to a terminal of the capacitor C32. Another terminal of the capacitance C32 is coupled to the power supply 710 (for example, the negative terminal).

In this embodiment, the third high-power switching unit 724_3 includes switching switches T34 to T37, and there are parasitic diodes D34 to D37 between respective first terminals and second terminals of the switching switches T34 to T37. In this embodiment, the first terminal of the switching switch T34 is coupled to the third charge and discharge unit 723_3, the second terminal of the switching switch T34 is coupled to the first output terminal P31, and the control terminal of the switching switch T34 is coupled to the third micro control unit 721_3. The first terminal of the switching switch T35 is coupled to the first output terminal P31, the second terminal of the switching switch T35 is coupled to the power supply 710 (for example, the negative terminal), and the control terminal of the switching switch T35 is coupled to the third micro control unit 721_3. The first terminal of the switching switch T36 is coupled to the third charge and discharge unit 723_3, the second terminal of the switching switch T36 is coupled to the second output terminal P32, and the control terminal of the switching switch T36 is coupled to the third micro control unit 721_3. The first terminal of the switching switch T37 is coupled to the second output terminal P32, the second terminal of the switching switch T37 is coupled to the power supply 710 (for example, the negative terminal), and the control terminal of the switching switch T37 is coupled to the third micro control unit 721_3. The first output terminal P21 and the second output P22 may be coupled to the third coil.

In this embodiment, the control switches T31, T33 and the switching switches T34 to T37 may be high-power components respectively. The control switches T31, T33 and the switching switches T34 to T37 may be N-type transistors, and the control switch T22 may be a P-type transistor. The above-mentioned first terminal and the second terminal may be the source terminal and the drain terminal respectively, and the above-mentioned control terminal may be the gate terminal. In this embodiment, the third micro control unit 721_3 may output multiple control signals to the control terminals of the control switches T31 to T33 and the switching switches T34 to T37. Specifically, when the power supply 710 supplies power to charge the capacitors C31, C32 in the third charge and discharge unit 723_3, since the parasitic diode D32 causes the voltage (Vgs) between the gate terminal and the source terminal of the control switch T32 to form a negative voltage, the control switch T32 may be turned on. Also, when the third charge and discharge unit 723_3 is to discharge, the third micro control unit 721_3 may control the control switch T31 to turn off, so that the gate terminal of the control switch T32 forms an open or non-connected state, so that the control switch T32 may be made in a non-conducting state. Therefore, the current released by the third charge and discharge unit 723_3 can be effectively prevented from flowing back to the power supply 710 or other circuit units. In this embodiment, the capacitors C31, C32 in the third charge and discharge unit 723_3 may provide the current to the switching switches T34 to T37. In this regard, the third micro control unit 721_3 may switch the switching switches T34 to T37 at high speeds to perform turning on and off, so that the third pulse current signal is output from the first output terminal P31 and the second output P32 to the third coil, so that the third coil can generate the corresponding electric field according to the third pulse current signal.

In this embodiment, the respective pulse repetition rates of the first pulse current signal, the second pulse current signal, and the third pulse current signal may be in a range of 1 to 250 pulses per second (PPS). The respective pulse intervals of the first pulse current signal, the second pulse current signal, and the third pulse current signal may be in a range of 1 to 120 seconds. The respective quantities of pulses of the first pulse current signal, the second pulse current signal, and the third pulse current signal may be in a range of 1 to 2000. The respective quantities of pulse clusters of the first pulse current signal, the second pulse current signal, and the third pulse current signal in a range of 1 to 5. The first pulse current signal, the second pulse current signal, and the third pulse current signal may also be single-phase pulse current signals or bi-phase pulse current signals respectively.

In this embodiment, the first micro control unit 721_1, the second micro control unit 721_2, and the third micro control unit 721_3 may control at least one of the first high-power switching unit 724_1, the second high-power switching unit 724_2, and the third high-power switching unit 724_3 to output the pulse current signal to at least one of the first coil, the second coil, and the third coil, so that at least one of the first coil, the second coil, and the third coil can generate an electric field. In this way, in the application scenarios of transcranial magnetic stimulation in FIG. 1A to FIG. 4, the electric field generated by the combination of at least one of the first coil, the second coil, and the third coil can generate the corresponding transcranial magnetic stimulation electric field to achieve effective transcranial magnetic stimulation function. In summary, the multi-channel coil control device of the disclosure can effectively drive

the multi-axis coil module by generating the multiple pulse current signals through the multi-channel coil control device, so that the multiple coils in the multi-axis coil module may be combined to generate the corresponding magnetic field, and the corresponding magnetic stimulation electric field can be generated at the treatment object (that is, an object located in the range of the magnetic field).

Although the disclosure has been disclosed above through embodiments, the embodiments are not intended to limit the disclosure. Persons with ordinary knowledge in the relevant technical field may make some changes and modifications without departing from the spirit and scope of the disclosure. Therefore, the protection scope of the disclosure shall be determined by the appended claims.

What is claimed is:

1. A multi-channel coil control device, comprising:
a power supply;
a multi-axis coil module; and
a multi-channel control circuit, coupled to the power supply and the multi-axis coil module, wherein the multi-channel control circuit comprises:
an anti-reverse flow module, coupled to the power supply;
a charge and discharge module, coupled to the anti-reverse flow module;
a high-power switching module, coupled to the charge and discharge module; and
a micro control module, coupled to the power supply, the anti-reverse flow module, the charge and discharge module, and the high-power switching module,
wherein the micro control module is configured to control the anti-reverse flow module, the charge and discharge module, and the high-power switching module to generate a plurality of pulse current signals according to a power signal provided by the power supply, so that the multi-axis coil module generates a magnetic field according to the plurality of pulse current signals.

2. The multi-channel coil control device as claimed in claim 1, wherein the multi-axis coil module comprises a plurality of coils, and the multi-channel control circuit outputs the plurality of pulse current signals to the plurality of coils to excite at least one of the plurality of coils to generate the magnetic field.

3. The multi-channel coil control device as claimed in claim 2, wherein the plurality of coils comprise a first coil, a second coil, and a third coil, and a first center axis of the first coil, a second center axis of the second coil, and a third center axis of the third coil are perpendicular to each other.

4. The multi-channel coil control device as claimed in claim 2, wherein the micro control module comprises a plurality of micro control units, and the plurality of micro control units are respectively coupled to the power supply, the anti-reverse flow module, the charge and discharge module, and the high-power switching module.

5. The multi-channel coil control device as claimed in claim 4, wherein the anti-reverse flow module comprises a plurality of anti-reverse flow units, the plurality of anti-reverse flow units are respectively coupled between the charge and discharge module and the plurality of micro control units, and the plurality of micro control units respectively control the plurality of anti-reverse flow units.

6. The multi-channel coil control device as claimed in claim 5, wherein the plurality of anti-reverse flow units comprise:
a first control switch, coupled to the micro control module;
a first resistor;
a second control switch, coupled to the first resistor, the power supply, and the charge and discharge module; and
a Zener diode, coupled between a first terminal and a second terminal of the second control switch, wherein the second control switch is a P-type transistor.

7. The multi-channel coil control device as claimed in claim 5, wherein the charge and discharge module comprises a plurality of charge and discharge units, the plurality of charge and discharge units are respectively coupled between the plurality of anti-reverse flow units and the high-power switching module, and the plurality of micro control units respectively control the plurality of charge and discharge units.

8. The multi-channel coil control device as claimed in claim 7, wherein the plurality of charge and discharge units respectively comprise:
a third control switch, coupled to the anti-reverse flow module and the micro control module;
a second resistor, coupled to the third control switch, the anti-reverse flow module, and the high-power switching module;
a first capacitor, coupled to the second resistor, the anti-reverse flow module, and the high-power switching module; and
a second capacitor, coupled to the third control switch, the second resistor, and the high-power switching module.

9. The multi-channel coil control device as claimed in claim 8, wherein the high-power switching module comprises a plurality of high-power switching units, the plurality of high-power switching units are respectively coupled between the plurality of charge and discharge units and the plurality of coils, and the plurality of micro control units respectively control the plurality of high-power switching units.

10. The multi-channel coil control device as claimed in claim 9, wherein the plurality of high-power switching units respectively comprise:
a first switching switch, coupled to the micro control module, the charge and discharge module, and a first output terminal;
a second switching switch, coupled to the micro control module, the charge and discharge module, and the first output terminal;
a third switching switch, coupled to the micro control module, the charge and discharge module, and a second output terminal; and
a fourth switching switch, coupled to the micro control module, the charge and discharge module, and the second output terminal.

11. The multi-channel coil control device as claimed in claim 1, wherein a pulse repetition rate of each of the plurality of pulse current signals is of 1 to 250 pulses per second (PPS).

12. The multi-channel coil control device as claimed in claim 1, wherein a pulse interval of each of the plurality of pulse current signals is of 1 to 120 seconds.

13. The multi-channel coil control device as claimed in claim 1, wherein a quantity of pulses of each of the plurality of pulse current signals is of 1 to 2000.

14. The multi-channel coil control device as claimed in claim 1, wherein a quantity of pulse clusters of each of the plurality of pulse current signals is of 1 to 5.
